(19) United States
(12) Patent Application Publication
(10) Pub. No.: US 2026/0137662 A1
(43) Pub. Date: May 21, 2026

(54) CD11B AGONISTS FOR TREATMENT OF AUTOIMMUNE DISEASE

(71) Applicants: Rush University Medical Center, Chicago, IL (US); 149 Bio, LLC, Miami, FL (US)

(72) Inventors: Vineet GUPTA, Chicago, IL (US); Xiabo LI, Chicago, IL (US); Veronica VILLANUEVA, Chicago, IL (US); Billy NGUYEN, Chicago, IL (US); Antonio BARBOSA, Miami, FL (US)

(73) Assignees: Rush University Medical Center, Chicago, IL (US); 149 Bio, LLC, Miami, FL (US)

(21) Appl. No.: 19/120,990

(22) PCT Filed: Oct. 16, 2023
(86) PCT No.: PCT/US2023/035247

§ 371 Date: Apr. 14, 2025

Related U.S. Application Data
(60) Provisional application No. 63/416,436, filed on Oct. 14, 2022.

(51) Int. Cl. A61K 31/427; A61P 17/14; A61P 37/06; G01N 33/68

(52) U.S. Cl. CPC A61K 31/427; A61P 17/14; A61P 37/06; G01N 33/6866; G01N 33/6869; G01N 33/6872; G01N 33/6893; G01N 2333/4703; G01N 2333/525; G01N 2333/5412; G01N 2333/545; G01N 2333/565; G01N 2333/70596; G01N 2800/24; G01N 2800/52

(57) ABSTRACT

Described herein are novel methods for the prevention and/or treatment of autoimmune diseases after disease onset associated with CD11b/CD18 activity, such as systemic lupus erythematosus (SLE), comprising administering a therapeutically effective amount of CD11b agonist LA1 ((Z)-4-(5-((3-benzyl-4-oxo-2-thioxothiazolidin-5-ylidene)methyl)furan-2-yl)benzoic acid) or it's choline salt.

### CROSS-REFERENCE TO RELATED APPLICATION

This application claims the benefit of U.S. Provisional Application No. 63/416,436 entitled “CD11B Agonists for Treatment of Autoimmune Disease,” filed Oct. 14, 2022, the entire contents of which are hereby incorporated by reference in their entirety.

### FEDERALLY SPONSORED RESEARCH

This invention was made with government support under R01DK084195 awarded by the National Institute of Health. The government has certain rights in the invention.

### TECHNICAL FIELD

The present disclosure provides a novel method of preventing or treating an autoimmune condition or disease in a patient.

### BACKGROUND OF THE INVENTION

Systemic lupus erythematosus (SLE) is a systemic autoimmune disease affecting multiple organs with various clinical manifestations. The disease has a strong gender bias, with 9 out of every 10 SLE patients being women. Over 40% of SLE patients develop some form of glomerular injury including lupus nephritis (LN). LN is a debilitating comorbidity of SLE and has become a predictor of morbidity and mortality. Current treatments for lupus nephritis include corticosteroids and immunosuppressants, which only lead to remission in a fraction of patients while causing serious complications. Thus, targeted, efficacious therapies are an unmet medical need that is urgently needed.

SLE is characterized by increased anti-double-stranded DNA (anti-dsDNA) antibodies, anti-RNA binding protein (anti-RNP) antibodies, and inflammatory cytokines which increase leukocyte activation. The resulting immune complexes are deposited on the glomerular capillaries, leading to the recruitment and activation of leukocytes contributing to glomerular injury. The pro-inflammatory milieu promotes a feed-forward loop causing further injury.

Genome-wide association studies have linked over 100 genetic risk loci to increased SLE risk. Further investigation identified single nucleotide polymorphisms (SNPs) on several immune response genes. Three coding region SNPs on the integrin alpha M (ITGAM) gene correlate with increased SLE and LN risk. ITGAM encodes CD11b, the alpha chain of the heterodimer CD11b/CD18 (Mac-1, CR3, alphaMbeta2), which is highly expressed on leukocytes. Function of the CD11b/CD18 integrin is regulated through conformational changes which is a major consequence of these SNPs. These conformation-altering SNPs appear to reduce integrin activation, ligand binding, cell adhesion, phagocytosis, and catch-bond formation without altering integrin surface expression. Previous studies have linked these mutations to reduced IFN-b in SLE patients and have shown that activation of CD11b mediates IFN-b and TLR-dependent inflammatory signaling (Faridi et al., 2017).

Urokinase plasminogen activator receptor (uPAR) is a GPI-anchored protein consisting of three domains encoded in humans by the PLAUR gene, cleavage of this receptor results in increased soluble uPAR (suPAR) in circulation. Previous studies have shown that suPAR on podocytes promotes foot process effacement and proteinuria through activation of integrin alpaVbeta3. suPAR was also found to activate beta3 integrins in podocytes leading to proteinuria and kidney pathology changes in mice. These studies have led to associations between uPAR, suPAR, and several disease states including several forms of kidney injury such as acute kidney injury (AKI), chronic kidney disease (CKD), and focal segmental glomerulosclerosis (FSGS). Few studies have investigated serum suPAR levels in patients with lupus nephritis (LN) caused by SLE, though several have identified it as a potential biomarker for disease onset.

The onset of SLE can be characterized by clinical symptoms in a patient such as skin rashes and lesions, alopecia, and in some cases, inflammation of the kidneys known as lupus nephritis (LN). Molecular indications of SLE can include proteinuria and increased levels of pro-inflammatory cytokines or other biomarkers in the blood or urine from a patient. These biomarkers can include IL-6, IL-1beta, IFNbeta, IFN I responsive gene signature, TNFalpha, uPAR, suPAR, NF-KB, or NLRP3. Previous studies have shown that a CD11b/CD18 integrin agonist can be effective at preventing the elevation of these biomarkers when the agonist is administered at the time of disease induction and thereby preventing disease onset and the development of clinical symptoms. SLE is generally diagnosed in a patient after disease onset when clinical symptoms are present. Therefore, there is a need for a treatment effective at not only preventing disease progression but also treating and reducing symptoms when administered after disease onset.

### SUMMARY

One embodiment described herein is a method of treating an autoimmune condition or disease in a patient in need thereof comprising: administering to the patient a therapeutically effective amount of a compound (Z)-4-(5-((3-benzyl-4-oxo-2-thioxothiazolidin-5-ylidene)methyl)furan-2-yl)benzoic acid (LA1), or its choline salt; wherein the compound is administered after disease onset. In one aspect, the method further comprises suppressing a Toll-like 7 receptor (TLR-7) pathway. In another aspect, the method further comprises lowering levels of one or more biomarkers in the patient, the biomarkers comprising IL-6, IL-1beta, IFNbeta, IFN I responsive gene signature, TNFalpha, uPAR, suPAR, NF-KB, or NLRP3. In one aspect of the method, the autoimmune condition or disease is systemic lupus erythematosus (SLE), a complication of lupus, or a disease associated with beta2 family of integrins. In another aspect of the method the treatment of SLE is dose dependent. In one aspect, the administration of the dose of the compound or choline salt is between about 0.1 mg/kg to about 1200 mg/kg. In another aspect, the dose is administered equally divided into one, two, three or four doses per day. In one aspect, the compound is administered to the patient after symptoms of an autoimmune disease are present.

In one aspect, the symptoms comprise skin lesions, inflammation, alopecia, or lupus nephritis. In another aspect of the method, after disease onset comprises about two to about four weeks.

In another embodiment described herein is a method for treatment in a patient in need thereof, of an autoimmune condition or disease in a patient in need thereof comprising: measuring levels of one or more biomarkers in the patient, wherein the levels of one or more markers are elevated relative to a healthy patient; administering to the patient an effective amount of a compound LA1, (Z)-4-(5-((3-benzyl-4-oxo-2-thioxothiazolidin-5-ylidene)methyl)furan-2-yl)benzoic acid, or its choline salt; and measuring levels of one or more markers in the patient; wherein the levels of one or more markers are lowered after administration. In one aspect, the method further comprises suppressing a Toll-like 7 receptor (TLR-7) pathway signaling. In one aspect, the biomarkers comprise IL-6, IL-1beta, IFNbeta, IFN I responsive gene signature, TNFalpha, uPAR, suPAR, NF-KB, or NLRP3. In one aspect, the autoimmune condition or disease is systemic lupus erythematosus (SLE), cutaneous lupus, a complication of lupus, or a disease associated with beta2 family of integrins. In another aspect, the levels of one or more biomarkers are lowered by an amount dependent on the amount of the compound administered. In another aspect, the administered dose of the compound or choline salt is between about 0.1 mg/kg to about 1200 mg/kg. In another aspect, the dose is administered in one, two, three or four, equal doses per day. In another aspect, the compound is administered to the patient between 2 and 4 weeks after disease onset. In another aspect, the administration to the patient is after symptoms of an autoimmune disease are present in the patient. In another aspect, the symptoms comprise skin lesions, inflammation, alopecia, or lupus nephritis.

Another embodiment described herein is a kit comprising: one or more reagents for measuring levels of one more biomarkers; a compound LA1, (Z)-4-(5-((3-benzyl-4-oxo-2-thioxothiazolidin-5-ylidene)methyl)furan-2-yl)benzoic acid, or its choline salt; wherein the markers comprise IL-6, IL-1beta, IFNbeta, IFN I responsive gene signature, TNFalpha, uPAR, suPAR, NF-KB, or NLRP3.

### BRIEF DESCRIPTION OF THE DRAWINGS

FIG. 1A illustrates levels of biomarker suPAR secretion from RAW 264.7 cells after 4 hours of treatment with cell culture medium (UT), LA1 (20 uM), LN patient sera (0.5%) in culture medium (LNsera), or co-treatment with LN sera (0.5%) and LA1 (20 uM). Bars show mean with SEM (n=3) (***P<0.001, **P<0.01, *P<0.05) .

FIG. 1B illustrates levels of biomarker TNFa secretion from RAW 264.7 cells after 4 hours of treatment with cell culture medium (UT), LA1 (20 uM), LN patient sera (0.5%) in culture medium (LNsera), or co-treatment with LN sera (0.5%) and LA1 (20 uM). Bars show mean with SEM (n=3) (***P<0.001, **P<0.01, *P<0.05) .

FIG. 2A illustrates the levels of biomarker suPAR secretion from RAW cells after treatment with LPS (50ng/ml), PMA (0.1 ug/mL), Pam2CSK4 (0.1 ug/mL), Pam3CSK4 (0.1 ug/mL), Imiquimod (IMQ) (20 ug/mL), LA1 (20 uM), or co-treatments. Bars show mean with SD (N=3) (*P<0.05, **P<0.01, ***P<0.001, ****P<0.0001).

FIG. 2B illustrates the levels of biomarker (A) suPAR and (B) TNF-a secretion from RAW cells after treatment with LPS (50ng/ml), PMA (0.1 ug/mL), Pam2CSK4 (0.1 ug/mL), Pam3CSK4 (0.1 ug/mL), Imiquimod (IMQ) (20 ug/mL), LA1 (20 uM), or co-treatments. Bars show mean with SD (N=3) (*P<0.05, **P<0.01, ***P<0.001, ****P<0.0001).

FIG. 2C illustrates relative gene expression of biomarker uPAR in RAW cells after 4 hours of treatment with Poly(I:C) (0.1 ug/mL), LA1 (20 uM), and Poly(I:C) or LA1 co-treatment.

FIG. 2D illustrates relative gene expression biomarker IL-1b in RAW cells after 4 hours of treatment with Poly(I:C) (0.1 ug/mL), LA1 (20 uM), or Poly(I:C) and LA1 co-treatment.

FIG. 3A illustrates transcriptional levels of biomarker IL-1b in myeloid cells after 4 hours of treatment with vehicle DMSO (UT), LA1 (20 uM), imiquimod (IMQ) (20 ug/mL), or IMQ and LA1 co-treatment. Bars show mean with SD (N=3) (*P<0.05, **P<0.01, ***P<0.001, ***P<0.0001).

FIG. 3B illustrates transcriptional levels of biomarker uPAR in myeloid cells after 4 hours of treatment with vehicle DMSO (UT), LA1 (20 uM), imiquimod (IMQ) (20 ug/mL), or IMQ and LA1 co-treatment. Bars show mean with SD (N=3) (*P<0.05, **P<0.01, ***P<0.001, ***P<0.0001).

FIG. 3C illustrates transcriptional levels of biomarker IL-6 in myeloid cells after 4 hours of treatment with vehicle DMSO (UT), LA1 (20 uM), imiquimod (IMQ) (20 ug/mL), or IMQ and LA1 co-treatment. Bars show mean with SD (N=3) (*P<0.05, **P<0.01, ***P<0.001, ***P<0.0001).

FIG. 3D illustrates transcriptional levels of biomarker NLRP3 in myeloid cells after 4 hours of treatment with vehicle DMSO (UT), LA1 (20 uM), imiquimod (IMQ) (20 ug/mL), or IMQ and LA1 co-treatment. Bars show mean with SD (N=3) (*P<0.05, **P<0.01, ***P<0.001, ***P<0.0001).

FIG. 4A illustrates the relative gene expression of biomarker IL-1bin myeloid cells after 4 hour of treatment with vehicle DMSO (UT), LA1 (20 uM), LPS (50ng/ml), or LPS and LA1 co-treatment. Bars show mean with SD (n=3) (*P<0.05, **P<0.01, ***P<0.001, ****P<0.0001).

FIG. 4B illustrates the relative gene expression of biomarker IL-18 in myeloid cells after 4 hour of treatment with vehicle DMSO (UT), LA1 (20 uM), LPS (50ng/ml), or LPS and LA1 co-treatment. Bars show mean with SD (n=3) (*P<0.05, **P<0.01, ***P<0.001, ***P<0.0001).

FIG. 4C illustrates the relative gene expression of biomarker uPAR in myeloid cells after 4 hour of treatment with vehicle DMSO (UT), LA1 (20 uM), LPS (50ng/ml), or LPS and LA1 co-treatment. Bars show mean with SD (n=3) (*P<0.05, **P<0.01, ***P<0.001, ****P<0.0001).

FIG. 4D illustrates the relative gene expression of biomarker IL-6 in myeloid cells after 4 hour of treatment with vehicle DMSO (UT), LA1 (20 uM), LPS (50ng/ml), or LPS and LA1 co-treatment. Bars show mean with SD (n=3) (*P<0.05, **P<0.01, ***P<0.001, ****P<0.0001).

FIG. 5A illustrates the levels of biomarker IL-1b secretion in myeloid cells after 4 hours of treatment with vehicle DMSO (UT), LA1 (20 uM), Imiquimod (IMQ) (20 ug/mL), or IMQ and LA1 co-treatment. Bars show mean with SD (N=3) (*P<0.05, **P<0.01, ***P<0.001, ***P<0.0001).

FIG. 5B illustrates the levels of biomarker suPAR secretion in myeloid cells after 4 hours of treatment with vehicle DMSO (UT), LA1 (20 uM), Imiquimod (IMQ) (20 ug/mL), or IMQ and LA1 co-treatment. Bars show mean with SD (N=3) (*P<0.05, **P<0.01, ***P<0.001, ***P<0.0001).

FIG. 5C illustrates the levels of biomarker IL-6 secretion in myeloid cells after 4 hours of treatment with vehicle DMSO (UT), LA1 (20 uM), Imiquimod (IMQ) (20 ug/mL), or IMQ and LA1 co-treatment. Bars show mean with SD (N=3) (*P<0.05, **P<0.01, ***P<0.001, ***P<0.0001).

FIG. 5D illustrates the levels of biomarker TNFalpha secretion in myeloid cells after 4 hours of treatment with vehicle DMSO (UT), LA1 (20 uM), Imiquimod (IMQ) (20 ug/mL), or IMQ and LA1 co-treatment. Bars show mean with SD (N=3) (*P<0.05, **P<0.01. ***P<0.001, ****P<0.0001).

FIG. 5E illustrates the levels of biomarker IFN-b secretion in myeloid cells after 4 hours of treatment with vehicle DMSO (UT), LA1 (20 uM), Imiquimod (IMQ) (20 ug/mL), or IMQ and LA1 co-treatment. Bars show mean with SD (N=3) (*P<0.05, **P<0.01, ***P<0.001, ****P<0.0001).

FIG. 6A illustrates gene expression of PLAUR in myeloid cells treated with NFkB inhibitor BMS 345541 and then subsequently treated for 8 hours with LA 1(20 uM), LPS (12.5, 25, 50, 100, or 250 ng/ml), or LPS and LA1 co-treatment (L+L).

FIG. 6B illustrates gene expression of NLRP3 in myeloid cells treated with NFkB inhibitor BMS 345541 and then subsequently treated for 8 hours with LA 1(20 uM), LPS (12.5, 25, 50, 100, or 250 ng/ml), or LPS and LA1 co-treatment (L+L).

FIG. 6C illustrates gene expression of and IL-6 in myeloid cells treated with NFkB inhibitor BMS 345541 and then subsequently treated for 8 hours with LA 1(20 uM), LPS (12.5, 25, 50, 100, or 250 ng/ml), or LPS and LA1 co-treatment (L+L).

FIG. 7A illustrates levels of biomarker IL-6 secretion in bone marrow derived macrophages (BMDMs) from C57BL/6 wild type (WT) mice, mice with global deletion of CD11b (KO), or C57BL/6 mice expressing CD11b with a point mutation (KI), the macrophages treated for 4 hours with vehicle DMSO (UT), LA1 (20 uM), IMQ (20 ug/mL), or IMQ and LA1 co-treatment. Bars show mean with SD (N=3) (*P<0.05, **P<0.01, ***P<0.001, ****P<0.0001).

FIG. 7B illustrates levels of biomarker IL-1b secretion in bone marrow derived macrophages (BMDMs) from C57BL/6 wild type (WT) mice, mice with global deletion of CD11b (KO), or C57BL/6 mice expressing CD11b with a point mutation (KI), the macrophages treated for 4 hours with vehicle DMSO (UT), LA1 (20 uM), IMQ (20 ug/mL), or IMQ and LA1 co-treatment. Bars show mean with SD (N=3) (*P<0.05, **P<0.01, ***P<0.001, ****P<0.0001).

FIG. 7C illustrates levels of biomarker suPAR secretion in bone marrow derived macrophages (BMDMs) from C57BL/6 wild type (WT) mice, mice with global deletion of CD11b (KO), or C57BL/6 mice expressing CD11b with a point mutation (KI), the macrophages treated for 4 hours with vehicle DMSO (UT), LA1 (20 uM), IMQ (20 ug/mL), or IMQ and LA1 co-treatment. Bars show mean with SD (N=3) (*P<0.05, **P<0.01, ***P<0.001, ****P<0.0001).

FIG. 7D illustrates levels of biomarker IFNb secretion in bone marrow derived macrophages (BMDMs) from C57BL/6 wild type (WT) mice, mice with global deletion of CD11b (KO), or C57BL/6 mice expressing CD11b with a point mutation (KI), the macrophages treated for 4 hours with vehicle DMSO (UT), LA1 (20 uM), IMQ (20 ug/mL), or IMQ and LA1 co-treatment. Bars show mean with SD (N=3) (*P<0.05, **P<0.01, ***P<0.001, ****P<0.0001).

FIG. 8A illustrates an experimental timeline for the measurement of suPAR levels, albumin creatinine ratios (ACR), anti-dsDNA antibody levels, and spleen size in wild type mouse models treated with IMQ, LA1, or IMO and LA1 co-treatment.

FIG. 8B illustrates levels of biomarker suPAR in the blood and urine of wild type mice (WT), mice treated with IMQ, mice co-treated with IMQ and vehicle DMSO or mice co-treated with IMQ and LA1 (2 mg/kg i.p. daily). Each dot represents a unique sample. Bars show mean +/−SEM (n=5).

FIG. 8C illustrates the albumin creatinine ratios (ACR) in the blood and urine of wild type mice (WT), mice treated with IMQ, mice co-treated with IMQ and vehicle DMSO or mice co-treated with IMQ and LA1 (2 mg/kg i.p. daily). Each dot represents a unique sample. Bars show mean +/−SEM (n=5).

FIG. 8D illustrates levels of anti-dsDNA antibodies in the blood and urine of wild type mice (WT), mice treated with IMQ, mice co-treated with IMQ and vehicle DMSO, or mice co-treated with IMQ and LA1 (2 mg/kg i.p. daily). Each dot represents a unique sample. Bars show mean +/−SEM (n=5).

FIG. 8E illustrates spleen size (mm) and weight (g) of wild type mice (WT), mice treated with IMQ, mice co-treated with IMQ and vehicle DMSO, or mice co-treated with IMQ and LA1. Each dot represents a unique sample. Bars show mean +/−SEM (n=5).

FIG. 9A illustrates an experimental timeline for the measurement of IL-1b levels, albumin creatinine ratios (ACR), anti-dsDNA antibody levels, and spleen size in wild type mice (WT), WT mice treated with IMQ, C57BL/6 mice expressing CD11b with a point mutation (KI), or KI mice treated with IMQ.

FIG. 9B illustrates levels of biomarker IL-1b in the blood and urine of wild type mice (WT), WT treated with IMQ, C57BL/6 mice expressing CD11b with a point mutation (KI), and KI mice treated with IMQ. Each dot represents a unique sample. Bars show mean +/−SEM (n=5).

FIG. 9C illustrates the albumin creatinine ratios (ACR) in the blood and urine of wild type mice (WT), WT treated with IMQ, C57BL/6 mice expressing CD11b with a point mutation (KI), and KI mice treated with IMQ. Each dot represents a unique sample. Bars show mean +/−SEM (n=5).

FIG. 9D illustrates levels of anti-dsDNA antibodies in the blood and urine of wild type mice (WT), WT treated with IMQ, C57BL/6 mice expressing CD11b with a point mutation (KI), and KI mice treated with IMQ. Each dot represents a unique sample. Bars show mean +/−SEM (n=5).

FIG. 9E illustrates spleen size (mm) and weight (g) of wild type mice (WT), WT mice treated with IMQ, C57BL/6 mice expressing CD11b with a point mutation (KI), and KI mice treated with IMQ. Each dot represents a unique sample. Bars show mean +/−SEM (n=5).

FIG. 10A illustrates the reduction of human dsDNA in a humanized SLE mouse model treated with LA1. NSG mice engrafted with healthy control or SLE PBMCs were subsequently treated with LA1 or vehicle and human dsDNA levels were measured after 4 weeks of treatment. Each dot represents a unique sample. Bars show mean +/−SEM (n=3-6).

FIG. 10B illustrates the reduction of suPAR in a humanized mouse model treated with LA1. NSG mice engrafted with healthy control or SLE PBMCs were subsequently treated with LA1 or vehicle and suPAR levels were measured after 4 weeks of treatment. Each dot represents a unique sample. Bars show mean +/−SEM (n=3-6).

FIG. 10C illustrates the reduction of IFN-B in a humanized mouse model treated with LA1. NSG mice engrafted with healthy control or SLE PBMCs were subsequently treated with LA1 or vehicle and IFN-B levels were measured after 4 weeks of treatment. Each dot represents a unique sample. Bars show mean +/−SEM (n=3-6).

FIG. 10D illustrates the reduction of albumin creatinine ratios (ACR) in a humanized mouse model treated with LA1. NSG mice engrafted with healthy control or SLE PBMCs were subsequently treated with LA1 or vehicle and ACR was measured after 4 weeks of treatment. Each dot represents a unique sample. Bars show mean +/−SEM (n=3-6).

FIG. 11 illustrates the concentration of biomarker IL-6 in wild type mice and wild type mice co-treated with various dose amounts of LPS and LA1 (0, 2, 10, 30, or 60 mg/kg). Each dot represents a unique sample. Bars show mean +/−SEM (n=4).

FIG. 12A illustrates levels of anti-dsDNA antibodies measured weekly in mice co-treated with IMQ and LA1 (30 mg/kg, BID) for 8 weeks. Each dot represents mean +/−SEM (n=4).

FIG. 12B illustrates levels of anti-dsDNA antibodies measured weekly in mice treated with IMQ for 8 weeks starting at week 0 and additionally treated with LA1 (30 mg/kg, BID) for 6 weeks, starting at week 2. Each dot represents mean +/−SEM (n=4).

FIG. 12C illustrates levels of anti-dsDNA antibodies measured weekly in mice treated with IMQ for 8 weeks starting at week 0 and additionally treated with LA1 (30 mg/kg, BID) for 4 weeks, starting at week 4. Each dot represents mean +/−SEM (n=4).

FIG. 12D illustrates levels of biomarker suPAR measured weekly in mice co-treated with IMQ and LA1 (30 mg/kg, BID) for 8 weeks. Each dot represents mean +/−SEM (n=4).

FIG. 12E illustrates levels of biomarker suPAR measured weekly in mice treated with IMQ for 8 weeks starting at week 0 and additionally treated with LA1 (30 mg/kg, BID) for 6 weeks, starting at week 2. Each dot represents mean +/−SEM (n=4).

FIG. 12F illustrates levels of biomarker suPAR measured weekly in mice treated with IMQ for 8 weeks starting at week 0 and additionally treated with LA1 (30 mg/kg, BID) for 4 weeks, starting at week 4. Each dot represents mean +/−SEM (n=4).

FIG. 12G illustrates an experimental timeline for the measurement of anti-dsDNA antibody and suPAR levels in mice treated with IMQ and LA1 for 8 weeks, the LA1 treatment initiated at various time points.

FIG. 13A illustrates reduction in alopecia in Mrl/lpr mice upon treatment with LA1, either as a co-treatment or treatment after 2-weeks of disease onset.

FIG. 13B illustrates percentage of skin effected by alopecia on the nose area of untreated mice (Ctrl), mice treated with a vehicle (Veh), and mice treated with LA1 (LA1). The graph shows data for co-treatment and delayed treatment where treatment is administered 2 week after disease onset. Bars show mean +/−SEM (n=5). (**** p<0.0001, *** p<0.001, * p<0.05) .

FIG. 13C illustrates lesion size (mm) in untreated mice (Ctrl), mice treated with a vehicle (Veh), and mice treated with LA1 (LA1). The graph shows data for co-treatment and delayed treatment where treatment is administered 2 week after disease onset. Bars show mean +/−SEM (n=5). (* p<0.0001, *** p<0.001, * p<0.05).

FIG. 14A illustrates alopecia in control mouse models (Ctrl), mice engrafted with heathy PBMCs (HC), humanized SLE models treated with vehicle (Veh), and humanized SLE models treated with LA1. A graph shows the percentage of skin effected by alopecia on the nose area of control mice (Ctrl), mice engrafted with heathy PBMCs (HC), humanized SLE mice treated with vehicle (Veh), and humanized SLE mice treated with LA1 (LA1). Bars show mean +/−SEM (n=5). **** p<0.0001.

FIG. 14B illustrates levels of GR1-positive and CD11b-positive cells in the spleens of control mice (Ctrl), humanized SLE mice treated with vehicle (SLE/LN+Veh), and humanized SLE mice treated with LA1 (SLE/LN+LA1). (*, p<0.05, **, p<0.01).

FIG. 14C illustrates the number of neutrophils in the kidney of control mice (Ctrl), mice engrafted with heathy PBMCs (HC), humanized SLE mice treated with vehicle (SLE/LN+Veh), and humanized SLE mice treated with LA1 (SLE/LN+LA1). (****, p<0.0001) .

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. As used herein, the articles “a,” “an,” and “the” are used herein to refers to one or to more than one (i.e., to at least one) of the grammatical object of the article. By way of example, “an element” may mean one element or more than one element.

As used herein, the terms “about” or “approximately” refers to a quantity, level, value, number, frequency, percentage, dimension, size, amount, weight or length that varies by as much as 10, 9, 8, 7, 6, 5, 4, 3, 2 or 1 % to a reference quantity, level, value, number, frequency, percentage, dimension, size, amount, weight or length. In some embodiments, the terms “about” or “approximately” when preceding a numerical value indicates the value plus or minus a range of 10%, 5%, or 1%.

The terms “comprise”, “comprises”, and “comprising” will be understood to imply the inclusion of a stated step or element or group of steps or elements but not the exclusion of any other step or element or group of steps or elements. By “consisting of” is meant to include, and be limited to, whatever follows the phrase “consisting of.” Thus, the phrase “consisting of” indicates that the listed elements are required or mandatory, and that no other elements may be present. By “consisting essentially of” is meant including any elements listed after the phrase, and be limited to other elements that do not interfere with or contribute to the activity or action specified in the disclosure for the listed elements.

The term “administer” refers to the direct application of an agent or compound to a subject. In embodiments, a medication is administered by ingestion, inhalation, infusion, injection, or any other means, whether self-administered or administered by a clinician or other medical professional.

As used herein, “an effective amount” refers to an amount that causes relief of symptoms of a disorder or disease as noted through clinical testing and evaluation, patient observation, and/or the like. An “effective amount” may further designate a dose that causes a detectable change in biological or chemical activity. The detectable changes may be detected and/or further quantified by one skilled in the art for the relevant mechanism or process. Moreover, an “effective amount” may designate an amount that maintains a desired physiological state, i.e., reduces or prevents significant decline and/or promotes improvement in the condition of interest. An “effective amount” may further refer to a “therapeutically effective amount”.

As used herein, the terms “individual”, “subject”, or “patient” are often used interchangeably and refer to any human or non-human mammal that may be treated with the methods disclosed herein. Suitable subjects (e.g., patients) include humans and non-human mammals (such as a rodent). Non-human primates and human patients are included. In one embodiment, subjects may include human patients that have been diagnosed with systemic lupus erythematosus. As used herein, the term “patient” refers to a subject that may receive a treatment of a disease or condition.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, “treatment”, “treat”, and “treating” refers to reversing, alleviating, mitigating, or slowing the progression of, or inhibiting the progress of, a disorder or disease or symptoms associated with such disorder or disease, and as described in more detail herein.

As used herein, “prevention”, “prevent”, and “preventing” refers to reducing the likelihood of developing a disease, disorder, or condition as described herein.

As used herein, “onset” and “disease onset” refers to the initial existence of symptoms of a disease or a change in the health of a patient.

As used herein, “LA1” and “compound” refers to the compound (Z)-4-(5-((3-benzyl-4-oxo-2-thioxothiazolidin-5-ylidene)methyl)furan-2-yl)benzoic acid.

As used herein, “salt” refers to a base addition salt prepared by combining LA1 free acid with a pharmaceutically acceptable base. As used herein, “choline salt” refers to LA1 salt prepared with choline as the counter ion.

The present disclosure is directed to a novel method of preventing and/or treating an autoimmune condition or disease in a patient. Specifically, the method comprises administering a therapeutically effective amount of a CD11b/CD18 agonist, such as LA1 or its choline salt, to a patient with an autoimmune disease. In some aspects, LA1 or its choline salt is administered to a patient to prevent or treat the autoimmune disease systemic lupus erythematosus (SLE). Thus, one embodiment described herein is a method of treating an autoimmune condition or disease in a patient in need thereof comprising administering to the patient an effective amount of a compound LA1, (Z)-4-(5-((3-benzyl-4-oxo-2-thioxothiazolidin-5-ylidene)methyl)furan-2-yl)benzoic acid, or its choline salt; wherein the compound is administered after between 2 to 4 week after disease onset. LA1 belongs to a class of compounds termed “leukadherins” for their ability to target integrin CD11b/CD18 and thereby reduce leukocyte activation and pro-inflammatory signaling pathways (Maiguel, et al., 2011; Faridi, et al. 2017).

In some aspects on the disclosure, the compound LA1 or its choline salt can be used to treat an autoimmune condition or disease, such as immune deficiency, acquired immune deficiency syndrome (AIDS), HIV, severe combined immunodeficiency, systemic lupus erythematosus (SLE), lupus, dermatitis herpetiformis, pemphigus vulgaris, vitililgo, scleroderma, polymyositis, dermatomyositis, spondyloarthropathies, Sjogren's syndrome, autoimmune myocarditis, autoimmune hemolytic anemia, pernicious anemia, autoimmune thrombocytopenia, autoimmune hepatitis, crohn's disease, ulcerative colitis, primary bililary cirrhosis, multiple sclerosis, myasthenia gravis, autoimmune neuropathies (Guillain-Barré), autoimmune uveitis, temporal arteritis, anti-phospholipid syndrome, vasculitides psoriasis, grave's disease, Hashimoto's thyroiditis, autoimmune oophoritis and orchitis, autoimmune polyendocrine syndrome (APS), cancer, leukemia, leukocyte adhesion deficiency, diabetes, hyper-IgM syndromes, rheumatoid arthritis, and autoimmune encephalomyelitis. In some aspects, the compound LA1 or its choline salt, can be used to target and activate beta2 family integrins, including CD11b/CD18 integrin, and treat an autoimmune condition or disease associated with the activity of CD11b/CD18 integrin, such as systemic lupus erythematosus (SLE) or lupus.

In some aspects, LA1 or its choline salt can be used to treat and/or prevent, in a patient, the development of common clinical symptoms related to autoimmune conditions and diseases. Exemplary symptoms of autoimmune conditions are diseases include, without limitation, inflammation of various tissues and organs, fatigue, recurring fever, joint pain and swelling, digestive issues, and skin issues.

In some aspects, the methods described herein comprise the use of LA1 or its choline salt to modify the signaling pathways in cells. Such pathways include, without limitation, the NF-KB pathway, AKT pathway, MAPK pathway, cytokine receptor signaling pathways, or Toll-like receptor (TLR) signaling pathways, such as TLR4 and TLR7. Thus, in one aspect, further comprising suppressing a Toll-like 7 receptor (TLR-7) pathway signaling.

In certain embodiments, the biomarkers of the present disclosure are detectable in a plasma sample of a patient. Plasma-based biomarkers may be tested from blood samples of patients via methods such as mass spectrometry and immunoassay. As such, plasma testing of biomarkers allows for a less invasive and low-cost option for monitoring discase progression in a patient. The blood sample testing of the present disclosure avoids costly or painful procedures such as MRI, PET scans. In some aspects of the present disclosure, the plasma-based biomarkers are tested to identify and monitor disease progression in patients. A plasma sample may be pre-tested, i.e., tested before a treatment, to determine the level of at least one biomarker in the patient. The level of the at least one biomarker may be indicative of a disease state. An embodiment of a compound of the present disclosure may then be administered to the patient. In some embodiments, a second plasma sample of the patient may then be tested after treatment to determine the level of the at least one biomarker after administration of the compound. Testing may determine whether the level of the biomarker has changed as a result of administration of the compound, i.e., increased or decreased, depending on the biomarker tested, such increase or decrease may indicate therapeutic utility of the administered compound.

In some aspects, the methods described herein comprise the use of LA1 or its choline salt to reduce the levels of secreted biomarkers in the cells of a patient with an autoimmune disease such as SLE. Exemplary biomarkers indicative of SLE include, without limitation, IL-6, IL-1beta, IFNbeta, IFN I responsive gene signature, TNFalpha, uPAR, suPAR, NF-KB, or NLRP3. In some aspects, the compound LA1 or its choline salt can be used to lessen the severity of clinical symptoms or conditions related to SLE. Clinical symptoms of SLE include, but are not limited to, skin rashes and lesions, inflammation, alopecia, or lupus nephritis (LN).

In one aspect of the disclosed method, detection of an autoimmune disease in a patient can involve measuring the levels of various biomarkers in samples obtained from said patient. Samples may be obtained from biological tissue, including but not limited to blood, bone marrow, or urine. Levels of biomarkers in the sample can be measured by processes known in the art. In some aspects, the levels of biomarkers in a sample can be quantified by concentration and/or relative gene expression.

In another aspect, the effective amount is any amount required to demonstrate a therapeutic effect. The therapeutically effective dosage of the compound of the instant disclosure may readily be determined for treatment of the desired indication by a clinician. The amount of compound to be administered in the treatment of one of these conditions may vary widely according to such considerations the dosage unit employed, the mode of administration, the period of treatment, the age and sex of the patient treated, and the nature and extent of the condition treated. The specific initial and continuing dosage regimen for each patient will vary according to the nature and severity of the condition as determined by the attending diagnostician, the age of the patient, the diet of the patient, time of administration, route of administration, rate of excretion of the drug, drug combinations, and the like. The desired mode of treatment and number of doses of the compound or pharmaceutical composition of the present disclosure may be ascertained by those skilled in the art using conventional treatment tests.

According to the disclosed method, a compound, such as LA1 or its choline, may be administered to a patient using any amount and any route administration effective for treatment of an autoimmune disease. In certain embodiments, LA1 or its choline salt may be administered at dosage amounts of about 0.1 mg/kg to about 1200 mg/kg. In another aspect of the method, the dosage may in an amount of about 0.5 mg/kg to about 1000 mg/kg, about 1.0 mg/kg to about 750 mg/kg, about 5 mg/kg to about 500 mg/kg, about 10 mg/kg to about 250 mg/kg. In some dosage may be in an amount of about 0.1 mg/kg, about 0.5 mg/kg, about 1 mg/kg, about 5 mg/kg, about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, or about 1200 mg/kg, In another aspect, the dosage may be administered one or more times a day, two times a day, three times a day, four times a day, five times a day, to obtain the desired therapeutic effect. In some aspects, the method of treatment described herein is dose dependent. The exact amount required will vary from patient to patient depending on depending on size, age, general condition of the patient, the severity of the condition, the mode of administration, and the like. In certain embodiments, the dosage for a patient can be determined by allometrically scaling a dose effective in an animal model to a dose appropriate for the patient. The compound, LA1 or its choline salt, can be formulated in a dosage unit for ease of administration. Preferably, the choline salt of LA1 is formulated in a tablet and can be administered orally to a patient. A dosage unit may contain from about 1 mg to 1000 mg of the compound. The compound, LA1 or its choline salt, can be formulated with various pharmaceutically acceptable compounding ingredients, generally referred to as carriers or diluents, or suitable preservatives, as necessary or desired. Conventional procedures for formulating the compound in appropriate dosage units may be utilized.

In some aspects, LA1 or it choline salt can be administered to a patient at the time of disease onset to prevent the progression of disease. In other aspects, LA1 or its choline salt can be administered to a patient after disease onset to reverse the effects of disease in the patient. For example, the compound can be administered to a patient between 1 week after disease onset to 6 months after disease onset, about 2 week to about 5 months after disease onset, about 3 week to about 4 months after disease onset, about 3 week to about 4 months after disease onset, about 4 week to about 3 months after disease onset, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, or more, after disease onset. In some aspects, the compound is administered between about 1 week to about 6 weeks, about 2 weeks to about 4 weeks, or about 3 weeks to about 5 weeks, after disease onset. LA1 or its choline salt can be administered at any time after the onset of clinical symptoms associated with SLE to reduce the severity of said symptoms.

Kits are also provided, and comprise one or more reagents for detecting elevated levels of one or more SLE biomarkers in a patient. In some aspects, kits may further include instructions for use in accordance with the methods of this disclosure and instructions for generating a diagnosis. The kit may further comprise a treatment, such as LA1 or its choline salt and description of a treatment regimen suggested for patients with a diagnosis. In one aspect, is a kit comprising, (a) one or more reagents for measuring levels of one or more biomarkers; (b) a compound LA1, (Z)-4-(5-((3-benzyl-4-oxo-2-thioxothiazolidin-5-ylidene)methyl)furan-2-yl)benzoic acid, or its choline salt.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### EXAMPLES

### General Methods

In general, the practice of the present disclosure employs unless otherwise indicated, conventional techniques of cell and tissue culture, molecular biology, immunology, microbiology, genetics, and protein and nucleic acid chemistry. The following examples are included for illustrative purposes only and are not intended to limit the scope of the invention

### Example 1

Soluble uPAR as a biomarker of LN in Vitro and Impact of LA1 on suPAR.

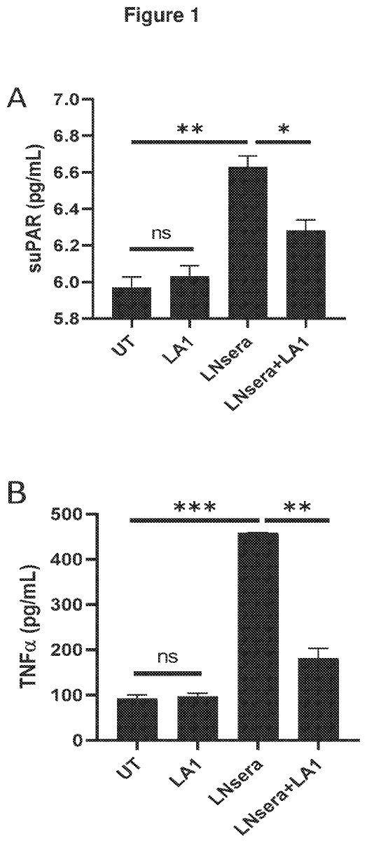

Myeloid cells from RAW 264.7 murine macrophage cell line were used to measure the concentration of pro-inflammatory biomarkers, soluble uPAR (suPAR) and TNFa, in the presence of lupus nephritis (LN) sera and LA1. The levels of pro-inflammatory biomarkers were measured in the supernatant of untreated cells, cells treated with LA1, cells treated with sera from patients with lupus nephritis (LN), and cells co-treated with LN sera and LA1. The concentrations of various pro-inflammatory biomarkers in cell supernatant were measured 4 hours after treatment using ELISA. FIG. 1A shows that treatment with LN sera resulted in significant upregulation of pro-inflammatory marker, soluble uPAR (suPAR), and that co-treatment with compound LA1 significantly reduced level of this marker in the cell supernatant. Similarly, FIG. 1B shows that treatment with LN sera resulted in significant upregulation of pro-inflammatory marker, TNFa, and that co-treatment with compound LA1 significantly reduced levels of this marker in the cell supernatant. The results suggest that elevated levels of suPAR and TNFa are a biomarkers for LN, caused by SLE, and LA1 can be useful in preventing the elevation of suPAR and TNFa levels at the onset of disease.

### Example 2

### Effect of LA1-Mediated CD11b Activation on TLR Signaling Pathways.

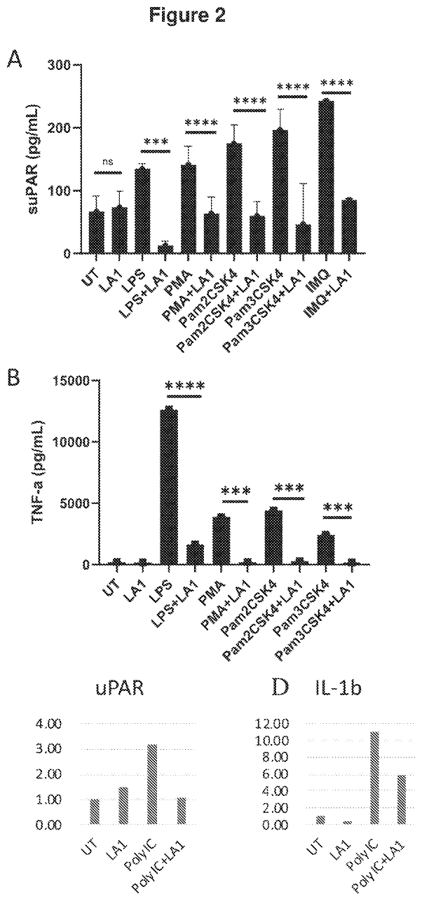

The effect of LA1 on TLR-stimulation dependent pro-inflammatory pathways was studied by stimulating myeloid cells with different TLR-stimulants (lipopolysaccharide (LPS), Pam2CSK 4, imiquimod (IMQ)) or with phorbol 12-myristate 13-acetate (PMA) and co-treating with LA1. The levels of suPAR (pg/mL) and TNFa (pg/mL) in cell supernatant were measured 4 hours after treatment using ELISA. The data in FIG. 2A shows increased levels of biomarker suPAR in the supernatant of cells treated with the various TLR-stimulants. Cells co-treated with a TLR-stimulant and LA1 had significantly reduced levels of biomarker suPAR. Similarly, the data in FIG. 2B shows increased levels of biomarker TNFa in the supernatant of cells treated with the various TLR-stimulants. Cells co-treated with a TLR-stimulant and LA1 had significantly reduced levels of biomarker TNFa. Relative gene expression was also assessed in myeloid cells treated with Poly(I:C), a known toll-like receptor (TLR) agonist, and co-treated with LA1. The results in FIG. 2C show significantly increased expression of biomarker uPAR, in the Poly(I:C) treated cells, which was significantly reduced upon co-treatment with LA1. The results in FIG. 2D show significantly increased expression of biomarker IL-1b in the Poly(I:C) treated cells, which was significantly reduced upon co-treatment with LA1. This suggests that activation of CD11b by LA1 suppresses several TLR signaling pathways.

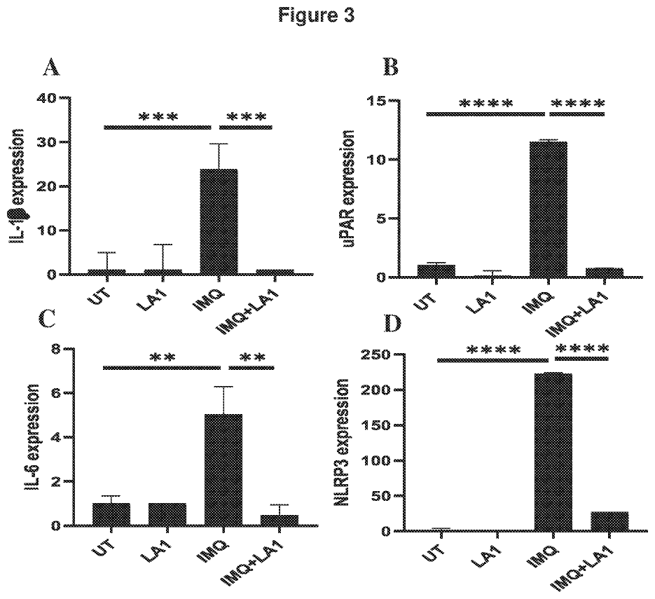

The impact of CD11b activation by LA1 on TLR7 mediated pathways was assessed by treating myeloid cells with imiquimod (IMQ), a known TLR7 stimulant, and co-treating myeloid cells with IMQ and LA1. Relative gene expression of various biomarkers was assessed by RT-PCR. As shown in FIGS. 3A-3D, TLR7 stimulation significantly increased expression of biomarkers IL-1b, and NLRP3. Co-treating with compound LA1 significantly reduced TLR7-dependent increases in uPAR, IL-6, IL-1b, and NLRP3 expression levels suggesting CD11b activation by LA1 reduces pro-inflammatory cytokine production at a transcriptional level.

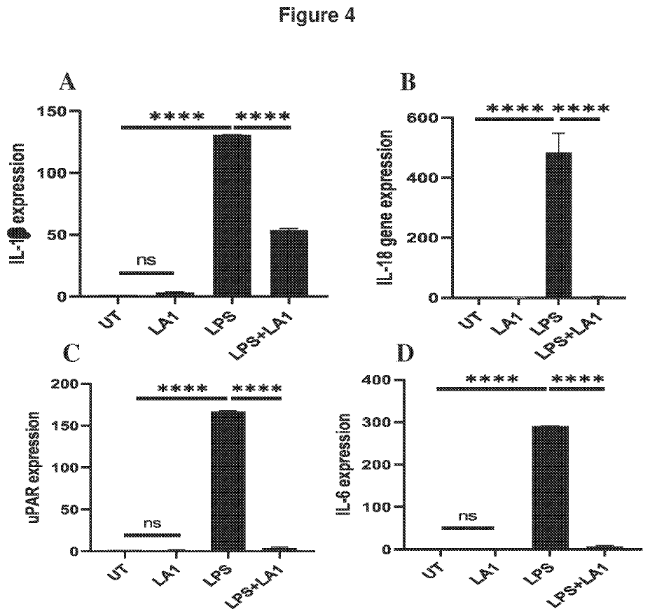

The impact of CD11b activation by LA1 on TLR4 mediated pathways was similarly assessed by stimulating myeloid cells with LPS, a known TLR4 stimulant, and co-treating with LA1. As shown in FIGS. 4A-4D, cells treated with TLR4 stimulant LPS showed significantly increased expression of biomarkers uPAR, IL-1b, IL-18, and IL-6, and the expression of those biomarkers was significantly decreased in cells co-treated with LA1. This further proports that CD11b activation by LA1 reduces pro-inflammatory cytokine production at a transcriptional level.

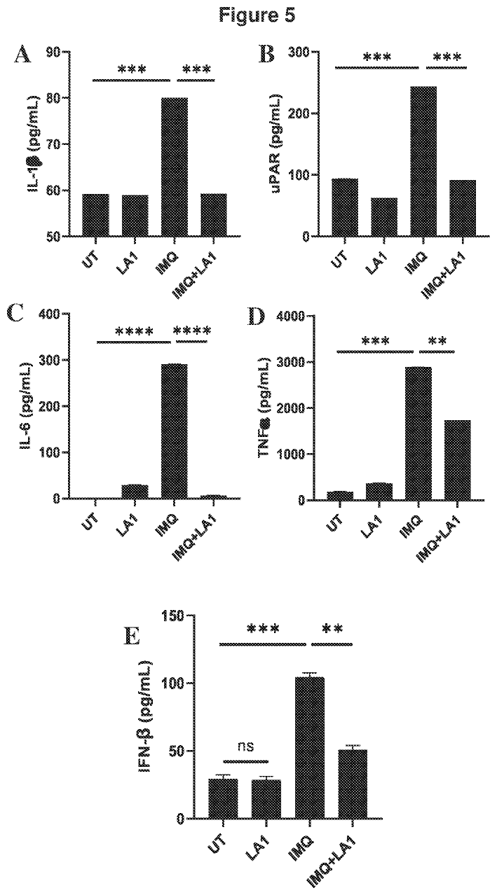

The concentration of TLR7 mediated pro-inflammatory biomarkers in untreated myeloid cells, myeloid cells treated with LA1, myeloid cells treated with IMQ, and myeloid cells co-treated with IMQ and LA1 was assessed by ELISA. FIGS. 5A-5E shows that TLR7 stimulation with IMQ significantly increased levels of pro-inflammatory cytokines uPAR, IL-6, IL-1b, IFN-b and TNF-a. Additionally, LA1 significantly reduced the TLR7-dependent increase in uPAR, IL-6, IL-1b, and TNF-a levels. This data further proports that CD11b activation reduces pro-inflammatory cytokine production. Treatment of cells with TLR4 agonist LPS and co-treatment with LA1 produced similar results.

### Example 3

Role of NFkB Pathway in uPAR Expression.

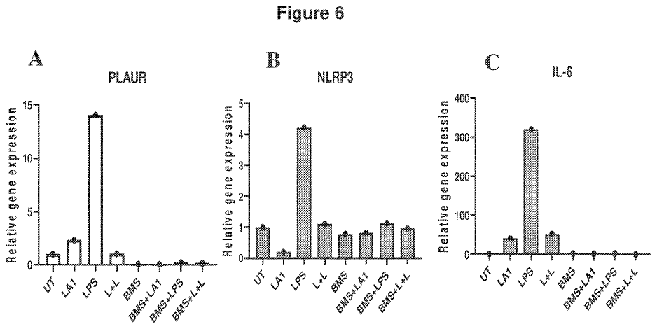

In order to determine potential molecular mechanisms resulting in uPAR expression, the association between uPAR/suPAR and other inflammatory cytokines and the NFkB pathway were investigated. uPAR expression levels in RAW 264.7 myeloid cells were evaluated in the absence or presence of a known NFkB inhibitor, BMS 345541. As shown in FIG. 6A, inhibition of NFkB led to reduced relative gene expression of PLAUR suggesting reduced uPAR expression. Similarly, FIG. 6B shows that expression levels of NLRP3 were reduced and FIG. 6C shows that expressions levels of IL-6 were reduced. These results suggest that NFkB plays a role in uPAR, IL-1b, and IL-6 expression.

### Example 4

### Effect of Genetic Activation of CD11b on Pro-Inflammatory TLR-Pathway in Myeloid Cells.

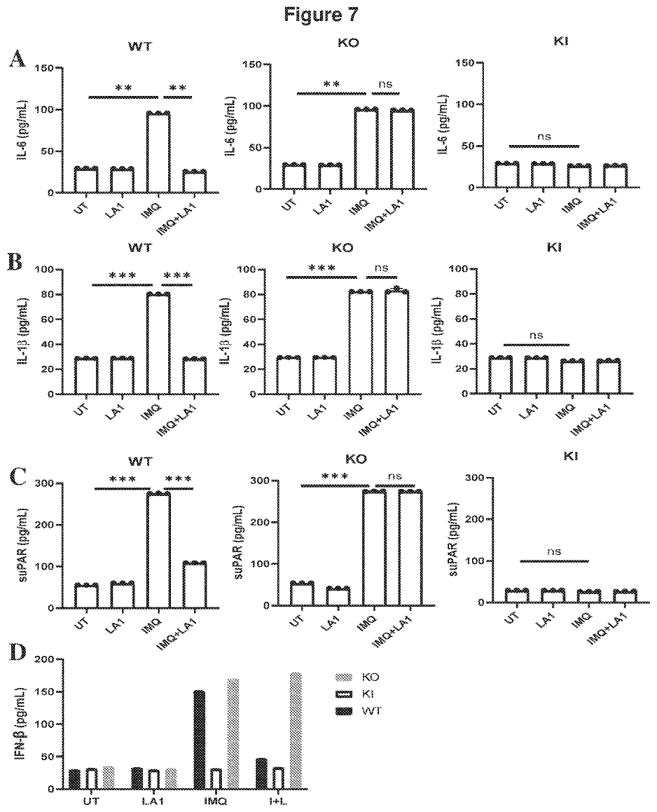

Bone marrow derived macrophages (BMDMs) from C57BL/6 wild type mice (WT), mice with global deletion of CD11b (KO), and C57BL/6 mice expressing CD11b with a point mutation (KI), as described previously (Martinez et al., 2020) were used to evaluate the effect of pharmacologic activation of CD11b by LA1 or genetic activation of CD11b on TLR-pathway dependent biomarkers. BMDMs from WT, KO, and KI mice were left untreated, treated with LA1, treated with IMQ, or co-treated with LA1 and IMQ and levels of biomarkers IL-1b, suPAR, IL-6 and IFN-b were measured 4 hours after treatment. As shown in FIGS. 7A-7D, treatment with IMQ resulted in elevated levels of biomarkers IL-1b, suPAR, IL-6 and IFN-b in cell supernatant from WT mice cells while co-treatment with IMQ and LA1 significantly suppressed these levels. KO cells also showed elevated levels of these markers with IMQ treatment, and co-treatment with LA1 had no effect, as the target CD11b is missing in these cells. Furthermore, cells expressing genetically activated CD11b (KI), did not show elevations in any of these markers and co-treatment with LA1 did not change basal levels of these mediators. These results show that pharmacologic (LA1) or genetic (KI) activation of CD11b suppresses pro-inflammatory TLR pathways in myeloid cells resulting in reduced levels of pro-inflammatory biomarkers.

### Example 5

### Effect of CD11b Activation by LA1 on ACR, Anti-dsDNA, and Spleen Size in IMQ Treated Mice.

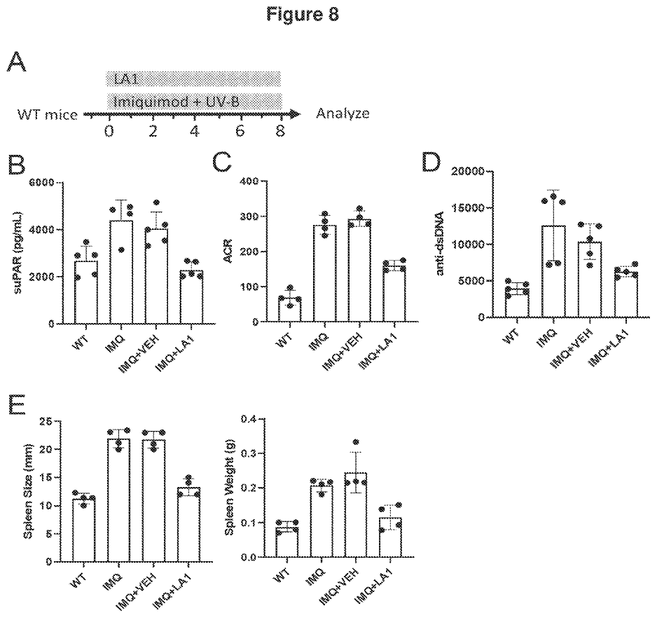

To investigate efficacy of LA1 as a therapeutic for SLE and autoimmune diseases in vivo, an IMQ-induced mouse model of lupus (Yokogawa et al, 2014) was used. Imiquimod (IMQ), a known TLR7 stimulant, was used to induce disease in wild type mice. Briefly, wild type C57BL/6 mice (WT) were treated topically with IMQ cream (Aldara) over the course of 8 weeks, as shown in the timeline of FIG. 8A, a group was also co-treated intraperitoneally with LA1 or vehicle DMSO. Blood and urine were collected for analysis of inflammatory cytokines and proteinuria over the course of treatment. As shown in FIGS. 8B-8D, mice treated with IMQ displayed significantly increased levels of anti-dsDNA antibodies, suPAR, and albumin creatinine ratios (ACR). Co-treatment with LA1 significantly reduced these markers, showing that activation of CD11b by LA1 reduces suPAR, proteinuria, and anti-dsDNA production. Additionally, spleen size and weight were recorded. IMQ treatment resulted in splenomegaly as shown in FIG. 8E, suggesting that IMQ treatment increases inflammation resulting in increased spleen size and weight. As shown, activation of CD11b by LA1 reduced spleen size and weight in co-treated mice suggesting that LA1 can be useful in preventing inflammation caused by the onset SLE.

### Example 6

Effect of IMQ treatment on ACR, Anti-dsDNA, and Spleen Size in KI Mice.

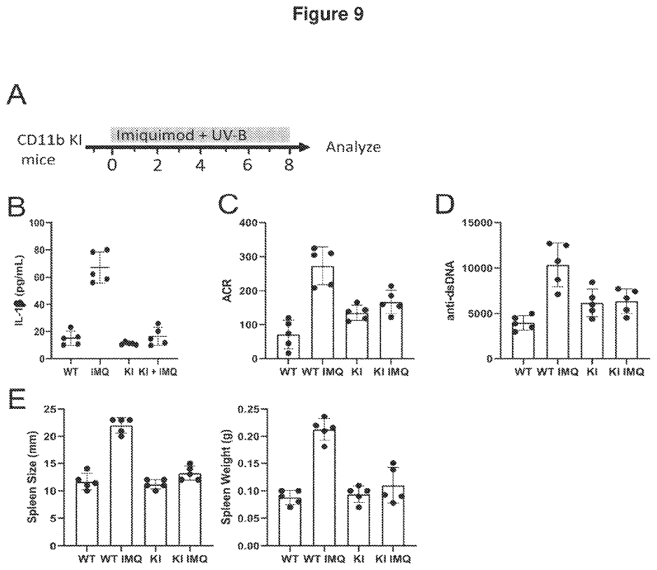

A genetic model of CD11b activation (KI) was utilized to independently study the role of CD11b activation in reducing SLE. A murine model that globally expresses a constitutively active CD11b through a genetically induced isoleucine 332 to glycine (I332G) mutation (Martinez et al., 2020) was used. Briefly, both C57BL/6 wild type (WT) and KI mice were treated topically with IMQ cream (Aldara) (Yokogawa et al., 2014) over the course of 8 weeks. Blood and urine were collected for analysis of inflammatory cytokines and proteinuria over the course of treatment. The timeline of treatment is illustrated in FIG. 9A. As shown in FIGS. 9B-9D, WT mice treated with IMQ displayed significantly increased levels of anti-dsDNA antibodies, suPAR, and albumin creatinine ratio (ACR) indicating inflammation and proteinuria. However, analyses of samples from the treated KI mice showed significantly reduced levels of IL-1b, and anti-dsDNA in the serum and significantly reduced urinary ACR. Additionally, as shown in FIG. 9E, the WT mice showed significant splenomegaly, while the KI mice did not, suggesting activation of CD11b significantly protected animals from kidney injury and inflammation in this IMQ-induced SLE model.

### Example 7

### Effect of CD11b Activation by LA1 on Alopecia, Pro-Inflammatory Markers and ACR in Humanized Lupus Mouse Model.

The efficacy of CD11b agonist LA1 as a therapeutic was studied using a generated humanized mouse model of SLE. To generate the humanized SLE model, immunocompromised NSG mice engrafted with peripheral blood mononuclear cells (PBMCs) from either SLE patients or from healthy human donors (control). 2.5×10⁶ purified PBMCs were injected intravenously into NSG mice on day 0. The humanized mice were monitored daily for significant clinical signs of SLE (alopecia, skin lesions, weight loss & arthritis), morbidity and mortality. Body weights were recorded weekly and skin lesions were photographed and scored weekly.

It was found that flow cytometry of mouse blood could confirm human PBMC engraftment in the immune-compromised mice, according to published protocols (Hahm et al., 2016). A small sample of peripheral blood was drawn from the mice under general anesthesia 2, 4 and 6 wks post-humanization by penetrating the retro-orbital sinus in mice with a sterile Pasteur pipette. The blood sample was split for fluorescence-activated cell sorting (FACS) analysis and serum preparation. For FACS staining, an anticoagulant was added to the blood, red blood cells were lysed, and the isolated leukocytes were stained with fluorescence-labeled antibodies against human CD45 and mouse CD45 for flow cytometry-based quantification to confirm high human cell engraftment rates. For serum, the whole blood was clotted at room temperature for 30 minutes, followed by a 10-minute centrifugation at 10,000 RPM. The red blood cell-free serum was transferred into micro-centrifuge tubes and stored at −80° C. until further analysis. Additionally, urine samples were collected weekly, transferred into a micro-centrifuge tube and stored at −80° for future analysis. Urine protein was measured using a sandwich ELISA specific for mouse albumin (Bethyl Laboratories, TX) and urinary creatinine was determined using the Creatinine Companion kit (Exocell, PA) according to manufacturer's protocol. It was found that mice engrafted with PBMCs from SLE patients (SLE-NSG) showed lupus-like clinical symptoms including increased inflammation, alopecia, and proteinuria.

To test the efficacy of LA1 on humanized SLE mouse models, the generated SLE-NSG mice were treated with LA1 (2 mg/kg i.p. daily) starting at two weeks post administration of PBMCs and until experiment completion. Concentrations of human dsDNA, mouse suPAR, and IFNb were measured in NSG mice, NSG mice engrafted with PBMCs from a healthy donor, untreated SLE-NSG mice, SLE-NSG mice treated with a vehicle, and SLE-NSG mice treated with LA1.

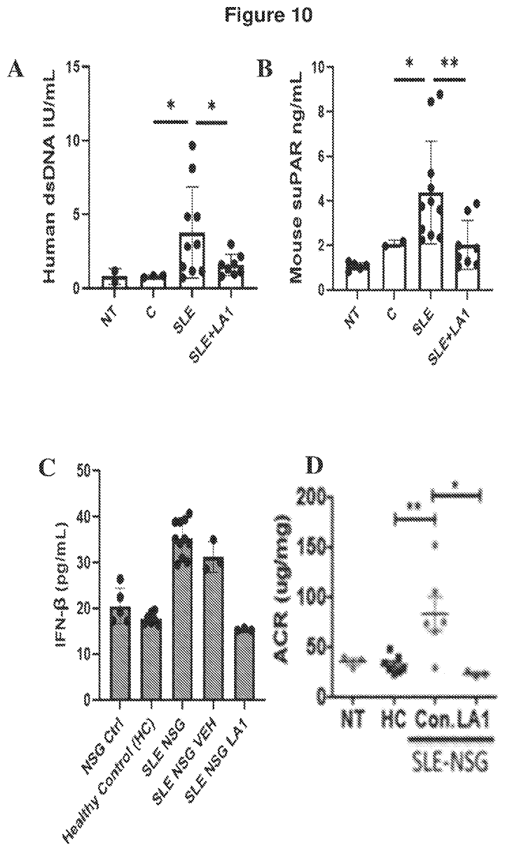

As shown in FIG. 10A-10D, SLE-NSG mice treated with LA1 showed significantly reduced concentrations of anti-double stranded DNA antibodies, suPAR, and IFNb in the serum and significantly reduced urinary albumin creatinine ratios (ACR) compared to SLE-NSG mice administered the vehicle alone. This shows that CD11b activation by LA1 reduces autoimmunity and proteinuria, thus reducing kidney damage in humanized models of SLE. Additionally, LA1 treatment resulted in significantly reduced alopecia in mice showing that LA1 can be effective in preventing the onset of clinical symptoms associated with SLE.

### Example 8

### Role of CD11b Activation by LA1 on Inflammatory IL-6 Levels.

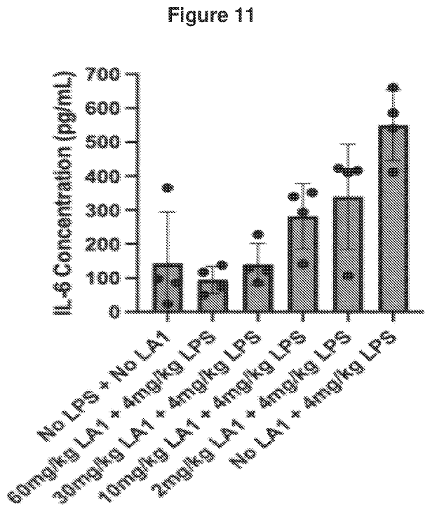

Administration of LPS to wild type C57BL/6 mice (WT) results in rapid elevation of pro-inflammatory mediators, such as IL-6, in murine sera. WT mice treated with LPS were used to study the dose-responsive efficacy of LA1 in reducing inflammatory cytokine levels in vivo. A single dose of LA 1 was orally administered to mice co-administered LPS. After 4 hours, sera was collected and used for ELISA. Data in FIG. 11 shows that LA1 significantly reduced IL-6 levels in murine sera, in a dose dependent fashion. Among the dose levels used, no difference in serum IL-6 levels was observed between non-LPS injected WT control and LPS injected animals administered LA1 at dose levels of 30 mg/kg and 60 mg/kg. These results suggest that the efficacy of LA1 as a treatment for SLE is dose dependent.

### Example 9

Effect of CD11b Activation by LA1 on anti-dsDNA Prophylactically and Post Disease Induction.

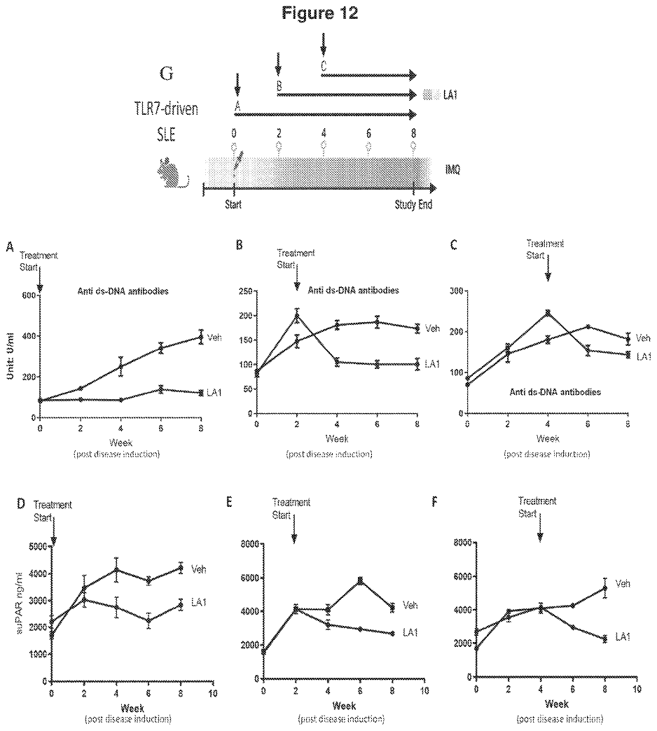

To assess therapeutic efficacy of LA1 in a TLR7-dependent SLE murine model, an IMQ-induced mouse model of lupus (Yokogawa et al., 2014) was used. To induce disease, wild type C57BL/ 6 mice (WT) were treated with IMQ cream (Aldara) over the course of 8 weeks. IMQ-induced mice were then treated with LA 1, the treatment initiated at 0, 2, and 4 weeks after disease induction, as shown in FIG. 12G. Anti-dsDNA antibody and suPAR levels were measured in the murine serum at various time points during treatment. As shown in FIG. 12A, treatment of LA1 significantly reduced anti-dsDNA antibody levels. Surprisingly, as shown in FIG. 12B, and 12C, treatment with LA1 two weeks or four weeks after the initial IMQ administration, and thus after the disease onset, also significantly reduced levels of anti-dsDNA antibodies in mouse sera. As shown in FIG. 12D, treatment of LA1 significantly reduced suPAR levels. Surprisingly, as shown in FIG. 12E, and 12F, treatment with LA1 two weeks or four weeks after the initial IMQ administration, and thus after the disease onset, also significantly reduced levels of suPAR in mouse sera. These results suggest that LA1 not only suppresses the elevation of biomarkers but also reduces the level biomarkers after elevation due to disease making it an effective treatment for autoimmune diseases related to CD11b activity, such as SLE.

### Example 10

### Effect of LA1 on Alopecia, Skin Lesions, GR1-Positive and CD11b-Positive Cell Levels, and Neutrophil Levels

To evaluate the efficacy of LA1 as a treatment for LN, MRL/ipr mouse models were used. MRL/lpr mice develop high circulating levels of IFN-I, systemic inflammation and multi-organ pathology, including skin, vascular, and kidney damage, and LN. Thus, this genetic mouse model mimics several features of human lupus and LN.

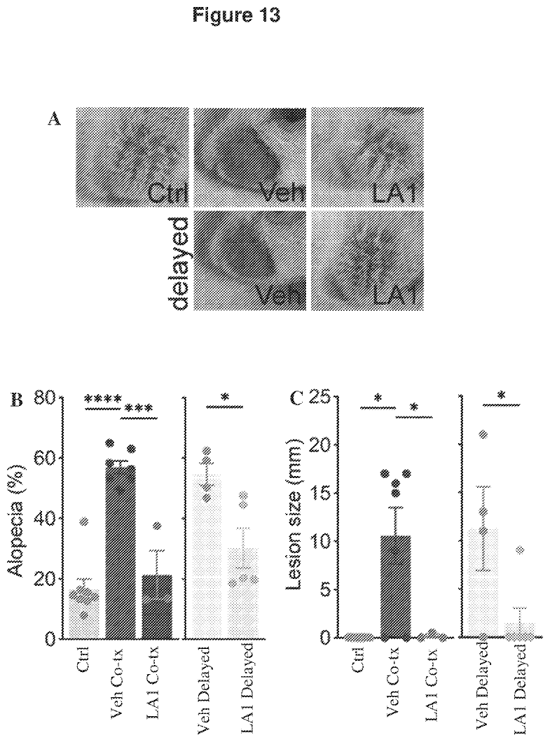

FIG. 13A illustrates a reduction in alopecia in Mrl/lpr mice upon treatment with LA1, either as a co-treatment or treatment after 2-weeks of disease onset. Images show a reduction in the severity of alopecia effecting the nose area upon when mice are treated with LA1. FIG. 13B shows a graph representing percentage of skin effected by alopecia in control Mrl/Mpj mice (Ctrl), Mrl/lpr mice treated with vehicle (Veh) and Mrl/lpr mice treated with LA1. FIG. 13C shows lesions size (mm) alopecia in control Mrl/Mpj mice (Ctrl), Mrl/lpr mice treated with vehicle (Veh) and Mrl/lpr mice treated with LA1. Results show data for mice administered treatment at the time of disease induction and also for mice administered treatment 2 week after disease onset. These results suggest that LA1 treatment reduces clinical symptoms of SLE, such as alopecia and skin lesions.

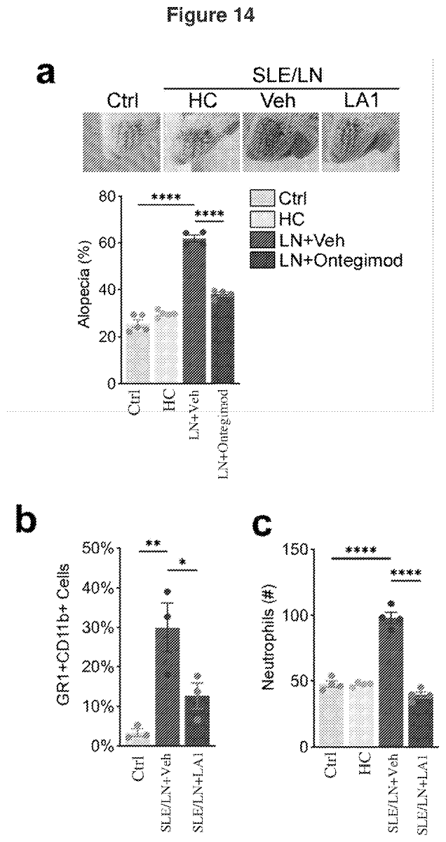

FIG. 14A shows images of alopecia in mouse nose area and a graph representing the quantification of alopecia in control mice (Ctrl), mice with PBMCs from healthy controls (HC), mice engrafted with PBMCs from lupus patients and treated with vehicle (Veh), and mice with PBMCs from lupus patients and treated with LA1. FIG. 14B shows the reduction in number of GR1-positive, CD11b-positive cells in spleens of mice from various treatment groups recited above. FIG. 14C shows the reduction in number of neutrophils infiltrating in the kidney, as determined by H&E staining of kidney sections from various animals from different treatment groups recited above. These results suggest that LA1 treatment reduces alopecia in humanized models of SLE.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

We claim:

1. A method of treating an autoimmune condition or disease in a patient in need thereof comprising:
administering to the patient a therapeutically effective amount of a compound (Z)-4-(5-((3-benzyl-4-oxo-2-thioxothiazolidin-5-ylidene)methyl)furan-2-yl)benzoic acid (LA1), or its choline salt;
wherein the compound is administered after disease onset.

2. The method of claim 1, further comprising suppressing a Toll-like 7 receptor (TLR-7) pathway.

3. The method of claim 1, further comprising lowering levels of one or more biomarkers in the patient, the biomarkers comprising IL-6, IL-1beta, IFNbeta, IFN I responsive gene signature, TNFalpha, uPAR, suPAR, NF-KB, or NLRP3.

4. The method of claims 1-3, wherein the autoimmune condition or disease is systemic lupus erythematosus (SLE), a complication of lupus, or a disease associated with beta2 family of integrins.

5. The method of claims 1-4, wherein the treatment of SLE is dose dependent.

6. The method of claims 1-5, wherein the administered dose of the compound or choline salt is between about 0.1 mg/kg to about 1200 mg/kg.

7. The method of claim 6, wherein the dose is administered equally divided into one, two, three or four doses per day.

8. The method of claim 6, wherein the compound is administered to the patient after symptoms of an autoimmune disease are present.

9. The method of claim 9, wherein the symptoms comprise skin lesions, inflammation, alopecia, or lupus nephritis.

10. The method of claim 1, wherein after disease onset comprises about 2 to about 4 weeks.

11. A method of treating an autoimmune condition or disease in a patient in need thereof comprising:
measuring levels of one or more biomarkers in the patient, wherein the levels of one or more markers are elevated relative to a healthy patient;
administering to the patient an effective amount of a compound LA1, (Z)-4-(5-((3-benzyl-4-oxo-2-thioxothiazolidin-5-ylidene)methyl)furan-2-yl)benzoic acid, or its choline salt; and
measuring levels of one or more markers in the patient;
wherein the levels of one or more markers are lowered after administration.

12. The method of claim 11, further comprising suppressing a Toll-like 7 receptor (TLR-7) pathway signaling.

13. The method of claim 11, wherein the biomarkers comprise IL-6, IL-1beta, IFNbeta, IFN I responsive gene signature, TNFalpha, uPAR, suPAR, NF-KB, or NLRP3.

14. The method of claims 11-13, wherein the autoimmune condition or disease is systemic lupus erythematosus (SLE), cutaneous lupus, a complication of lupus, or a disease associated with beta2 family of integrins.

15. The method of claims 11-14, wherein the levels of one or more biomarkers are lowered by an amount dependent on the amount of the compound administered.

16. The method of claims 11-15, wherein the administered dose of the compound or choline salt is between about 0.1 mg/kg to about 1200 mg/kg.

17. The method of claim 16, wherein the dose is administered in one, two, three or four, equal doses per day.

18. The method of claim 16, wherein the compound is administered to the patient between 2 and 4 weeks after disease onset.

19. The method of claim 16, wherein the compound is administered to the patient after symptoms of an autoimmune disease are present in the patient.

20. The method of claim 19, wherein the symptoms comprise skin lesions, inflammation, alopecia, or lupus nephritis.

21. A kit comprising:
one or more reagents for measuring levels of one more biomarkers;
a compound LA1, (Z)-4-(5-((3-benzyl-4-oxo-2-thioxothiazolidin-5-ylidene)methyl)furan-2-yl)benzoic acid, or its choline salt;
wherein the markers comprise IL-6, IL-1beta, IFNbeta, IFN I responsive gene signature, TNFalpha, uPAR, suPAR, NF-KB, or NLRP3.
